(19) United States
(12) Patent Application Publication
(10) Pub. No.: US 2005/0181116 A1
(43) Pub. Date: Aug. 18, 2005

(54) Method for coating a medical device using a matrix assisted pulsed-laser evaporation technique and associated system and medical device

(76) Inventor: Rob Worsham, Somerville, MA (US)

Correspondence Address: KENYON & KENYON, 1500 K STREET NW, SUITE 700, WASHINGTON, DC, 20005, US

(21) Appl. No.: 10/782,056

(22) Filed: Feb. 18, 2004

(51) Int. Cl. A61L 2/00; B05D 3/00; C23C 8/00

(52) U.S. Cl. 427/2.1; 427/585

(57) ABSTRACT

A method is provided for coating at least a portion of at least one medical device. The method includes arranging the at least one medical device in a vapor cone and directing an energy beam at a frozen target. The frozen target includes an agent and the energy beam vaporizes the agent into the vapor cone. A device is provided for coating at least one medical device. The device includes a target assembly, an energy beam directed at the target assembly, and an arrangement adapted to hold the at least one medical device in a vapor cone. The vapor cone originates at a target point that an energy beam beam contacts a frozen target in the target assembly. A medical device is provided having a coating applied by a method. The method includes arranging the medical device in a vapor cone and directing an energy beam at a frozen target. The frozen target includes an agent and the energy beam vaporizes the agent into the vapor cone.

### FIELD OF THE INVENTION

The present invention relates to the manufacturing of medical devices. More particularly, the present invention relates to a device and method for coating medical devices using a Matrix Assisted Pulsed-Laser Evaporation (MAPLE) technique.

### BACKGROUND INFORMATION

Medical devices may be coated so that the surfaces of such devices have desired properties or effects. For example, it may be useful to coat medical devices to provide for the localized delivery of therapeutic agents to target locations within the body, such as to treat localized disease (e.g., heart disease) or occluded body lumens. Localized drug delivery may avoid some of the problems of systemic drug administration, which may be accompanied by unwanted effects on parts of the body which are not to be treated. Additionally, treatment of the afflicted part of the body may require a high concentration of therapeutic agent that may not be achievable by systemic administration. Localized drug delivery may be achieved, for example, by coating balloon catheters, stents and the like with the therapeutic agent to be locally delivered. The coating on medical devices may provide for controlled release, which may include long-term or sustained release, of a bioactive material.

Aside from facilitating localized drug delivery, medical devices may be coated with materials to provide beneficial surface properties. For example, medical devices are often coated with radiopaque materials to allow for fluoroscopic visualization during placement in the body. It is also useful to coat certain devices to achieve enhanced biocompatibility and to improve surface properties such as lubriciousness.

Coatings have been applied to medical devices by processes such as dipping, spraying, vapor deposition, plasma polymerization, and electrodeposition. Although these processes have been used to produce satisfactory coatings, they have numerous, associated potential drawbacks. For example, it may be difficult to achieve coatings of uniform thicknesses, both on individual parts and on batches of parts. Further, many conventional processes require multiple coating steps or stages for the application of a second coating material, or to allow for drying between coating steps or after the final coating step.

There is, therefore, a need for a cost-effective method of coating medical devices that results in uniform, defect-free coatings and uniform drug doses per unit device. The method would allow for a multiple stage coating in order to apply a bioactive material that may be environmentally sensitive, e.g., due to heat and light (including ultra-violet) exposure. Multiple stage coating may also be used to prevent degradation of the bioactive material due to process-related forces (e.g., shear). The method would thus allow for better control of the sensitivity of the bioactive material and reduce any potential degradation due to environmental issues. The method would also reduce variations in the coating properties.

Current coating techniques may result in thicker coatings, resulting in excess bioactive ingredient being deposited on the medical device. Excessive bioactive ingredient delivered to the lumen may be toxic. Thinner coatings may allow more precise deposition of bioactive ingredient(s) on the medical appliance, and may allow greater precision in the delivery of the bioactive agent. Therefore, an efficient method of applying thin coats of materials to medical devices is desired.

The MAPLE technique has been used to provide thin coatings. “The deposition, structure, pattern deposition, and activity of biomaterial thin-films by matrix-assisted pulsed-laser evaporation (MAPLE) and MAPLE direct write,” in Thin Solid Films (volumes 398-399, November 2001, pages 607-614), discusses the MAPLE process and is incorporated herein by reference.

### SUMMARY

According to an exemplary method of the present invention, medical devices are coated using a Matrix Assisted Pulsed-Laser Evaporation (MAPLE) technique. An energy beam is directed at a frozen target including a drug and polymer suspended in a solution which may be frozen. The frozen target may be arranged on a refrigerated rotating assembly. The energy beam may be directed at the frozen target and vaporize the target into a vapor cone. A medical device may be placed in the vapor cone and may be situated close to the frozen target. The vaporized target may include the drug/polymer combination and the solvent. The vaporized material may deposit in a controlled fashion on the target, and may deposit at a slow rate. The solvent may evaporate from the medical device and may be transported out of a vacuum chamber by a pump. A secondary gas source may assist in delivering the vaporized coating from the target to the medical device.

A device for coating at least one medical device includes a target assembly adapted to hold a frozen target and an energy beam directed at the frozen target being held by the target assembly. The device also includes an arrangement adapted to hold the at least one medical device in a vapor cone. The frozen target includes an agent. The vapor cone originates at a target point that an energy beam pulse from the energy beam contacts the frozen target.

A medical device having a coating applied by a method. The method includes directing an energy beam at a frozen target and vaporizing by the energy beam the frozen target into a vapor cone. The method also includes arranging the medical device in the vapor cone.

### BRIEF DESCRIPTION OF THE DRAWINGS

FIG. 1 illustrates schematically an exemplary embodiment of a system using the MAPLE technique to coat a stent.

FIG. 2 is a flowchart illustrating an exemplary method according to the present invention.

### DETAILED DESCRIPTION

The MAPLE process may produce an advantageous degree of specificity, i.e., small areas of a medical device (for instance, the ends of a stent) may be coated to a separate product specification than the remainder of the stent. The MAPLE process may provide greater freedom in the selection of active agents due to fewer degradation effects in the active agent. The MAPLE process may provide an increased ability to control release-kinetics of the active agents due to the ability to control coating finish. The MAPLE process may allow greater freedom in the use of polymer substrates including those involving cross-linking and bonding of radicals.

The drug release kinetics may be controlled by either varying the degree of crosslinks or by varying the density of the finish on the substance. This may give some control of the micro-porosity of the coating and regulate the diffusion of the drug and/or active agent.

By using a high-energy technique such as MAPLE, this may allow for more use/manipulation of chemical bonding reactions such as cross-linking or free radical reactions that are not available with conventional coating techniques.

Certain conventional coating techniques may have degrading effects on an active agent. For example, a coating technique that demands a high temperature may denature proteins and therefore may not be used in coating a medical device with proteins. Therefore the MAPLE coating technique may allow certain active agents to be coated when other techniques may be less favorable.

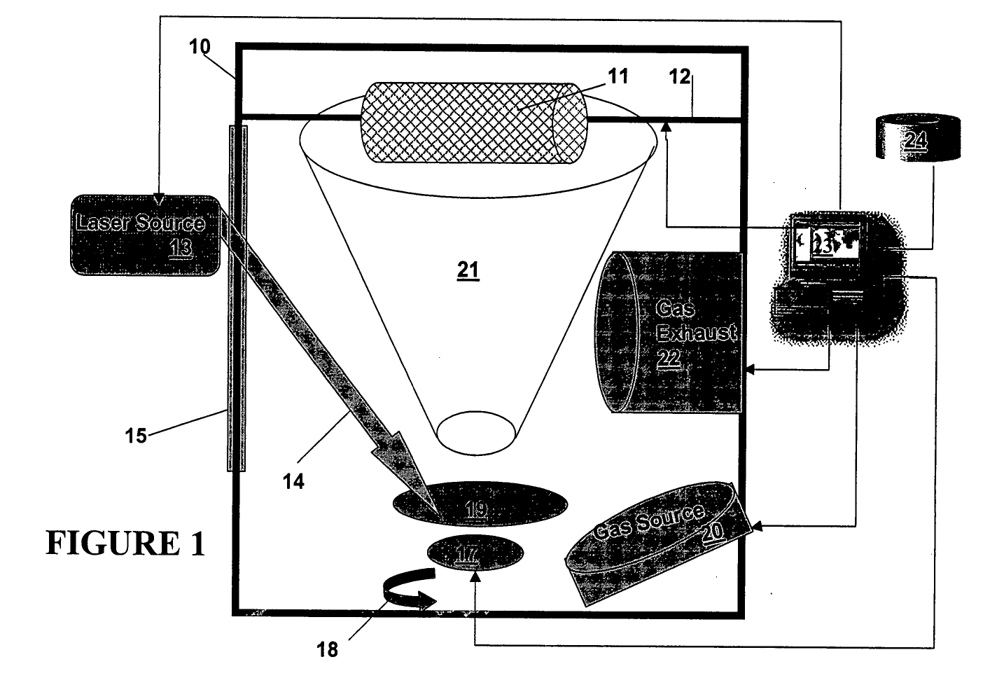

FIG. 1 includes vacuum chamber 10 enclosing stent 11 arranged on holder 12. Holder 12 may be adapted to move stent 11 laterally, longitudinally, vertically and/or rotatably. Holder 12 may be adapted to hold more than one stent, and may be adapted to move stent 11 out of vacuum chamber 10 and move another stent 11 into vacuum chamber 10. Holder 12 may be adapted to continuously move stent 11 and replace it with a new stent 11 in order to coat stent 11 in a continuous fashion rather than in a batch coating process.

Laser source 13 is situated outside vacuum chamber 10 in such a manner that it projects laser beam 14 through window 15 of vacuum chamber 10. Alternatively, laser source 13 may be situated inside vacuum chamber 10, and vacuum chamber 10 may or may not have window 15. Laser source 13 may be any type of laser emitting a laser beam and/or a laser pulse of any appropriate frequency. Laser beam 14 may possibly be a beam of ultraviolet (UV) light, or any other type of appropriate energy beam.

Laser beam 14 may impinge on target 19, which may be a frozen solution of a drug and polymer. The drug and polymer combination in the frozen solution of target 19 may be a therapeutic and/or bioactive agent useful for any number of purposes. Some of the possibilities for therapeutics and/or bioactive agents coated on a stent are discussed below. When laser beam 14 impinges on target 19, the laser may impart energy to the molecules in the frozen solution matrix, and may vaporize the solute, drug, and/or polymer. The evaporated material may eject from the surface of target 19 and may form vapor cone 21. Vapor cone 21 may include molecules of solute, drug, and/or polymer moving with some velocity from target 19 towards stent 11. The velocity of the molecules in vapor cone 21 may be provided solely by the vaporization of the frozen material of target 19 in the vacuum provided by vacuum chamber 10.

Additionally, there may be a pressure differential assisting the movement of molecules in vapor cone 21 which may be created by positioning a pump near the top of vacuum chamber 10 (for instance, gas exhaust 22). Alternatively, gas source 20 may be utilized to assist the movement, and/or increase the velocity, of molecules of solute, drug, and/or polymer moving from target 19 towards stent 11. Gas source 20 may provide a flow of an inert gas and/or a material that may not interfere with the drug, bioactive agent, and/or polymer being deposited on stent 11.

Target 19 may be situated on rotating refrigerated assembly 17. Rotating refrigerated assembly 17 may be refrigerated and thereby maintain target 19 in a frozen state. Additionally, rotating refrigerated assembly 17 may rotate to expose new areas of target 19 to laser beam 14, thereby enabling all of target 19 to be vaporized and utilized for coating stent 11. Alternatively, all of vacuum chamber 10 may be refrigerated to maintain target 19 in a frozen state. Additionally and alternatively, laser source 13 may redirect laser beam 14 to cause laser beam 14 to impinge on new areas of target 19. Additionally and alternatively, window 15 may operate to focus and redirect laser beam 14.

The molecules of solute, drug, and/or polymer moving from target 19 towards stent 11 may deposit on stent 11 molecule-by-molecule. The deposition of molecules may therefore be controlled and may enable thin layers to be deposited. The solute in the vapor may deposit on stent 11, but may subsequently evaporate again into vacuum chamber 10. Evaporated solute may be removed from vacuum chamber 10 by gas exhaust 22 (which may be an air pump). Gas exhaust 22 may enable vacuum chamber 10 to operate continuously in a vacuum or near-vacuum state, thereby promoting the evaporation of deposited liquid solute from stent 11 or elsewhere in vacuum chamber 10.

Processor 23 may control any or all of holder 12, laser source 13, rotating refrigerated assembly 17, gas source 20, and gas exhaust 22. Processor 23 may be electrically coupled to memory 24, which may include process parameters for coating various types of medical devices with various types of drugs and bioactive agents.

Alternative exemplary embodiments may provide for additional lasers and/or additional targets for the deposition of multiple layers. Additionally, it may be possible to coat just a portion of stent 11 (for instance, the ends of stent 11), by appropriate positioning or moving of stent 11 in vapor cone 21. Additionally and alternatively, masks and/or other barriers may be utilized to promote the coating of a portion of stent 11, while maintaining another portion of stent 11 free of coating.

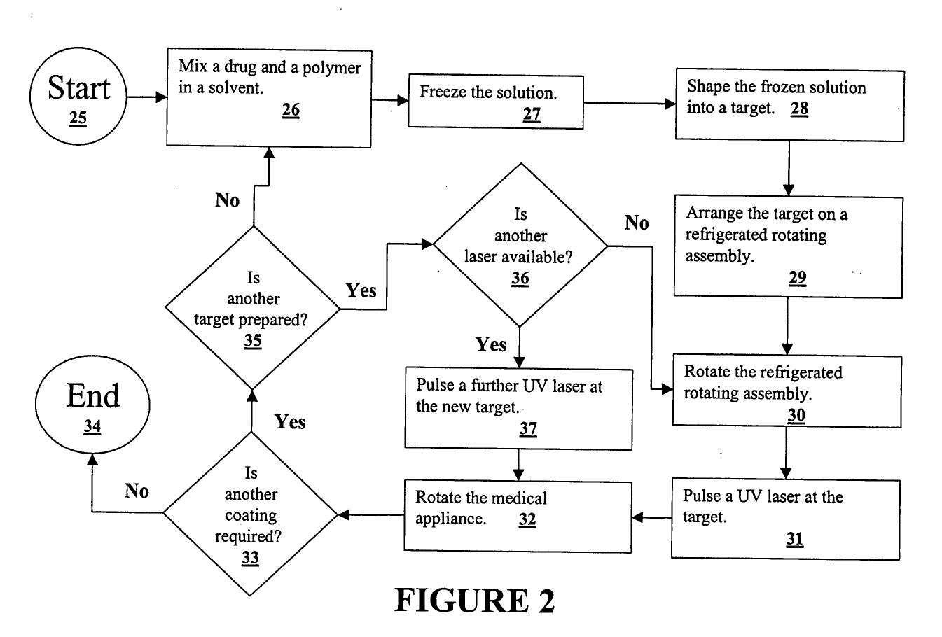

FIG. 2 is a flowchart illustrating an exemplary method according to the present invention. The flow in FIG. 2 begins in start circle 25 and proceeds to action 26, which indicates to mix a drug and a polymer in a solvent. From action 26 the flow proceeds to action 27, which indicates to freeze the solution. From action 27, the flow proceeds to action 28, which indicates to shape the frozen solution into a target. From action 28, the flow proceeds to action 29, which indicates to arrange the target on a refrigerated rotating assembly. From action 29, the flow proceeds to action 30, which indicates to rotate the refrigerated rotating assembly. From action 30, the flow proceeds to action 31, which indicates to pulse a UV laser at the target. From action 31, the flow proceeds to action 32, which indicates to rotate the medical appliance. From action 32, the flow proceeds to question 33, which asks whether another coating is required. If the response to question 33 is in the negative, the flow proceeds to end circle 34. If the response to question 33 is in the affirmative, the flow proceeds to question 35, which asks whether another target is prepared. If the response to question 35 is in the negative, the flow proceeds to action 26. If the response to question 35 is in the affirmative, the flow proceeds to question 36, which asks whether another laser is available. If the response to question 36 is in the negative, the flow proceeds to action 30. If the response to question 33 is in the affirmative, the flow proceeds to action 37, which indicates to pulse a further UV laser at the new target. From action 37, the flow proceeds to action 32.

Medical implants are used for innumerable medical purposes, including the reinforcement of recently re-enlarged lumens, the replacement of ruptured vessels, and the treatment of disease such as vascular disease by local pharmacotherapy, i.e., delivering therapeutic drug doses to target tissues while minimizing systemic side effects. Such localized delivery of therapeutic agents has been proposed or achieved using medical implants which both support a lumen within a patient's body and place appropriate coatings containing absorbable therapeutic agents at the implant location. Examples of such medical devices include catheters, guide wires, balloons, filters (e.g., vena cava filters), stents, stent grafts, vascular grafts, intraluminal paving systems, implants and other devices used in connection with drug-loaded polymer coatings. Such medical devices are implanted or otherwise utilized in body lumina and organs such as the coronary vasculature, esophagus, trachea, colon, biliary tract, urinary tract, prostate, brain, and the like.

The term “therapeutic agent” as used herein includes one or more “therapeutic agents” or “drugs”. The terms “therapeutic agents” and “drugs” are used interchangeably herein and include pharmaceutically active compounds, nucleic acids with and without carrier vectors such as lipids, compacting agents (such as histones), viruses (such as adenovirus, andenoassociated virus, retrovirus, lentivirus and α-virus), polymers, hyaluronic acid, proteins, cells and the like, with or without targeting sequences.

Specific examples of therapeutic agents used in conjunction with the present invention include, for example, pharmaceutically active compounds, proteins, cells, oligonucleotides, ribozymes, anti-sense oligonucleotides, DNA compacting agents, gene/vector systems (i.e., any vehicle that allows for the uptake and expression of nucleic acids), nucleic acids (including, for example, recombinant nucleic acids; naked DNA, cDNA, RNA; genomic DNA, cDNA or RNA in a non-infectious vector or in a viral vector and which further may have attached peptide targeting sequences; antisense nucleic acid (RNA or DNA); and DNA chimeras which include gene sequences and encoding for ferry proteins such as membrane translocating sequences (“MTS”) and herpes simplex virus-1 (“VP22”)), and viral, liposomes and cationic and anionic polymers and neutral polymers that are selected from a number of types depending on the desired application. Non-limiting examples of virus vectors or vectors derived from viral sources include adenoviral vectors, herpes simplex vectors, papilloma vectors, adeno-associated vectors, retroviral vectors, and the like. Non-limiting examples of biologically active solutes include anti-thrombogenic agents such as heparin, heparin derivatives, urokinase, and PPACK (dextrophenylalanine proline arginine chloromethylketone); antioxidants such as probucol and retinoic acid; angiogenic and anti-angiogenic agents and factors; anti-proliferative agents such as enoxaprin, angiopeptin, rapamycin, angiopeptin, monoclonal antibodies capable of blocking smooth muscle cell proliferation, hirudin, and acetylsalicylic acid; anti-inflammatory agents such as dexamethasone, prednisolone, corticosterone, budesonide, estrogen, sulfasalazine, acetyl salicylic acid, and mesalamine; calcium entry blockers such as verapamil, diltiazem and nifedipine; antineoplastic/antiproliferative/anti-mitotic agents such as paclitaxel, 5-fluorouracil, methotrexate, doxorubicin, daunorubicin, cyclosporine, cisplatin, vinblastine, vincristine, epothilones, endostatin, angiostatin and thymidine kinase inhibitors; antimicrobials such as triclosan, cephalosporins, aminoglycosides, and nitrofurantoin; anesthetic agents such as lidocaine, bupivacaine, and ropivacaine; nitric oxide (NO) donors such as linsidomine, molsidomine, L-arginine, NO-protein adducts, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, Warfarin sodium, Dicumarol, aspirin, prostaglandin inhibitors, platelet inhibitors and tick antiplatelet factors; vascular cell growth promotors such as growth factors, growth factor receptor antagonists, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogenous vascoactive mechanisms; survival genes which protect against cell death, such as anti-apoptotic Bcl-2 family factors and Akt kinase; and combinations thereof. Cells can be of human origin (autologous or allogenic) or from an animal source (xenogeneic), genetically engineered if desired to deliver proteins of interest at the insertion site. Any modifications are routinely made by one skilled in the art.

Polynucleotide sequences useful in practice of the invention include DNA or RNA sequences having a therapeutic effect after being taken up by a cell. Examples of therapeutic polynucleotides include anti-sense DNA and RNA; DNA coding for an anti-sense RNA; or DNA coding for tRNA or rRNA to replace defective or deficient endogenous molecules. The polynucleotides can also code for therapeutic proteins or polypeptides. A polypeptide is understood to be any translation product of a polynucleotide regardless of size, and whether glycosylated or not. Therapeutic proteins and polypeptides include as a primary example, those proteins or polypeptides that can compensate for defective or deficient species in an animal, or those that act through toxic effects to limit or remove harmful cells from the body. In addition, the polypeptides or proteins that can be injected, or whose DNA can be incorporated, include without limitation, angiogenic factors and other molecules competent to induce angiogenesis, including acidic and basic fibroblast growth factors, vascular endothelial growth factor, hif-1, epidermal growth factor, transforming growth factor α and β, platelet-derived endothelial growth factor, platelet-derived growth factor, tumor necrosis factor α, hepatocyte growth factor and insulin like growth factor; growth factors; cell cycle inhibitors including CDK inhibitors; anti-restenosis agents, including p15, p16, p18, p19, p21, p27, p53, p57, Rb, nFkB and E2F decoys, thymidine kinase (“TK”) and combinations thereof and other agents useful for interfering with cell proliferation, including agents for treating malignancies; and combinations thereof. Still other useful factors, which can be provided as polypeptides or as DNA encoding these polypeptides, include monocyte chemoattractant protein (“MCP-1”), and the family of bone morphogenic proteins (“BMP's”). The known proteins include BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are any of BMP-2, BMP-3, BMP-4, BMP-5, BMP-6 and BMP-7. These dimeric proteins can be provided as homodimers, heterodimers, or combinations thereof, alone or together with other molecules. Alternatively or, in addition, molecules capable of inducing an upstream or downstream effect of a BMP can be provided. Such molecules include any of the “hedgehog” proteins, or the DNA's encoding them.

Coatings used with the present invention may comprise a polymeric material/drug agent matrix formed, for example, by admixing a drug agent with a liquid polymer, in the absence of a solvent, to form a liquid polymer/drug agent mixture. Curing of the mixture typically occurs in-situ. To facilitate curing, a cross-linking or curing agent may be added to the mixture prior to application thereof. Addition of the cross-linking or curing agent to the polymer/drug agent liquid mixture possibly should not occur too far in advance of the application of the mixture in order to avoid over-curing of the mixture prior to application thereof. Over curing may be avoided in the method and device according to an exemplary embodiment of the present invention by virtue of the fact that the solution of drug and polymer may be frozen, which may thereby avoid the problem of overcuring.

Curing may also occur in-situ by exposing the polymer/drug agent mixture, after application to the luminal surface, to radiation such as ultraviolet radiation or laser light, heat, or by contact with metabolic fluids such as water at the site where the mixture has been applied to the luminal surface. In coating systems employed in conjunction with the present invention, the polymeric material may be either bioabsorbable or biostable. Any of the polymers described herein that may be formulated as a liquid may be used to form the polymer/drug agent mixture.

In an exemplary embodiment, the polymer used to coat the medical device is provided in the form of a coating on an expandable portion of a medical device. After applying the drug solution to the polymer and evaporating the volatile solvent from the polymer, the medical device may be inserted into a body lumen where it is positioned to a target location. In the case of a balloon catheter, the expandable portion of the catheter is subsequently expanded to bring the drug-impregnated polymer coating into contact with the lumen wall. The drug is released from the polymer as it slowly dissolves into the aqueous bodily fluids and diffuses out of the polymer. This may enable administration of the drug to be site-specific, limiting the exposure of the rest of the body to the drug.

Very thin polymer coatings may be possible according to an exemplary embodiment of the present invention. It is also within the scope of the present invention to apply multiple layers of polymer coating onto a medical device. Such multiple layers may be of the same or different polymer materials.

The polymer of the present invention may be hydrophilic or hydrophobic, and may be selected from the group consisting of polycarboxylic acids, cellulosic polymers, including cellulose acetate and cellulose nitrate, gelatin, polyvinylpyrrolidone, cross-linked polyvinylpyrrolidone, polyanhydrides including maleic anhydride polymers, polyamides, polyvinyl alcohols, copolymers of vinyl monomers such as EVA, polyvinyl ethers, polyvinyl aromatics, polyethylene oxides, glycosaminoglycans, polysaccharides, polyesters including polyethylene terephthalate, polyacrylamides, polyethers, polyether sulfone, polycarbonate, polyalkylenes including polypropylene, polyethylene and high molecular weight polyethylene, halogenated polyalkylenes including polytetrafluoroethylene, polyurethanes, polyorthoesters, proteins, polypeptides, silicones, siloxane polymers, polylactic acid, polyglycolic acid, polycaprolactone, polyhydroxybutyrate valerate and blends and copolymers thereof as well as other biodegradable, bioabsorbable and biostable polymers and copolymers. Coatings from polymer dispersions such as polyurethane dispersions (BAYHDROL®, etc.) and acrylic latex dispersions are also within the scope of the present invention. The polymer may be a protein polymer, fibrin, collagen and derivatives thereof, polysaccharides such as celluloses, starches, dextrans, alginates and derivatives of these polysaccharides, an extracellular matrix component, hyaluronic acid, or another biologic agent or a suitable mixture of any of these, for example. In one embodiment of the invention, the preferred polymer is polyacrylic acid, available as HYDROPLUS® (Boston Scientific Corporation, Natick, Mass.), and described in U.S. Pat. No. 5,091,205, the disclosure of which is hereby incorporated herein by reference. U.S. Pat. No. 5,091,205 describes medical devices coated with one or more polyisocyanates such that the devices become instantly lubricious when exposed to body fluids. In another preferred embodiment of the invention, the polymer is a copolymer of polylactic acid and polycaprolactone.

While the present invention has been described in connection with the foregoing representative embodiment, it should be readily apparent to those of ordinary skill in the art that the representative embodiment is exemplary in nature and is not to be construed as limiting the scope of protection for the invention as set forth in the appended claims.

1. A method for coating at least a portion of a stent, comprising:
providing a frozen target including a polymer and a therapeutic agent;
directing an energy beam at the frozen target;
vaporizing at least a portion of the frozen target with the energy beam into a vapor; and
contacting the stent with the vapor, thereby coating at least a portion of the stent with the polymer and therapeutic agent.

2. (canceled)

3. The method of claim 1, further comprising dissolving the therapeutic agent and polymer in a solvent to prepare a target solution, the target solution adapted to form the frozen target.

4. The method of claim 3, further comprising:
freezing the target solution to make the frozen target; and
mounting the frozen target on a refrigerated assembly.

5. The method of claim 4, wherein the refrigerated assembly is adapted to rotate.

6. The method of claim 3, further comprising:
enclosing the frozen target and the stent in a vacuum chamber; and
removing by a pump the solvent from the vacuum chamber after deposition of the polymer and therapeutic agent on the stent.

7. The method of claim 1, further comprising directing a gas flow to transport the vapor to the stent.

8. The method of claim 1, wherein the energy beam is pulsed.

9. The method of claim 1, further comprising directing at least one of the energy beam and another energy beam at another frozen target, the other frozen target including another agent, the at least one of the energy beam and the other energy beam vaporizing the other target into another vapor.

10-30. (canceled)

31. The method of claim 1, wherein another portion of the stent is not coated.

32. The method of claim 1, wherein another portion of the stent is coated with another agent.

33. The method of claim 1, wherein at least one end of the stent is coated.
